(19) United States
(12) Patent Application Publication
(10) Pub. No.: US 2011/0071601 A1
(43) Pub. Date: Mar. 24, 2011

(54) KEYBOARD WITH INTEGRATED ELECTROMAGNETIC RADIATION THERAPY

(75) Inventor: Paul P. Schneider, Bayside, WI (US)

(73) Assignee: RESTECHE LLC, Bayside, WI (US)

(21) Appl. No.: 12/565,170

(22) Filed: Sep. 23, 2009

(51) Int. Cl. A61N 5/06

(52) U.S. Cl. 607/88

(57) ABSTRACT

A device, such as a computer pointing device or a keyboard, that provides electromagnetic radiation therapy to help prevent or promote healing for repetitive stress injury. If the keyboard is used, one or more EMR-emitting devices, such as a plurality of LEDs, may be housed in the keyboard body and configured to emit EMR such as visible and/or infrared light within preferred wavelength ranges, to provide therapeutic affects for the wrist and hand of a user.

### CLAIM OF PRIORITY AND RELATED COPENDING APPLICATIONS

This application claims priority from U.S. Ser. No. 12/366,947 filed Feb. 6, 2009, and titled “Computer Peripheral With Integrated Electromagnetic Radiation Therapy”, which in turn claims priority from U.S. Ser. No. 11/462,217 filed on Feb. 7, 2008 and titled “Computer Peripheral With Integrated Infrared Therapy and Method of Making Same” each of which are hereby incorporated by reference in their entirety into this disclosure.

### BACKGROUND OF THE INVENTION

The present invention generally relates to a computer peripheral. More particularly, the invention relates to a keyboard providing electromagnetic radiation (EMR) therapy.

Computer peripherals such as mice, keyboards, joysticks, pen tablets, scanners and microphones are well known in the computer industry, and are commonly used in homes and offices. The motion of a computer mouse, for example, directs movement of a cursor on a computer screen. Typically, the user moves the computer mouse in two-dimensional motion relative to a surface, such as a desk. This motion can translate into the motion of a cursor on a computer screen.

An individual's use of a computer mouse, keyboard or other computer peripheral can lead to tissue damage that causes pain. The symptoms of individuals with maladies such as arthritis, tendonitis, Repetitive Strain Injury (RSI), or carpal tunnel syndrome can become exacerbated after sporadic or consistent use of such peripherals. Therapy for these injuries and maladies commonly requires halting the use of the computer peripheral while the therapy is applied.

EMR from various sources, such as light emitting diode (LED) technology, has the ability to deliver EMR deep into the human body's tissues, and is noninvasive and drug-free. LED laser light travels in one direction from its source in a highly columated and coherent beam, resulting in a higher concentration of light than from a light bulb. This allows the laser beam to penetrate more deeply into tissues living processes. Emitting LEDs at various wavelengths, intensities, and durations have been found to have a wide range of effects in the body including: bio-stimulation and regeneration, and increased nutrient transfer into cells; tissue healing; pain reduction; and decreased inflammation.

Although laser therapy has been around for more than 40 years, many rehabilitation professionals are just learning about the healing properties of this modality. As early as the 1960s, the effects of low-energy argon and helium-neon lasers on the behavior of biological tissues and cells were studied both in-vivo and in-vitro. In the following decade, data was presented on the positive effects of low-energy lasers for wound healing, pain relief and laser acupuncture. Based on these early findings, rehab specialists in Canada, Europe, Asia, Australia and New Zealand began incorporating low-energy laser therapy into the treatment of patients with musculoskeletal injuries and painful conditions. As laser use spread across the globe, it sparked published clinical evidence supporting its effectiveness.

Despite worldwide acceptance, it was not until the 1980s that low-energy infrared and helium-neon laser devices became available to physical therapists in the United States. Although laser devices were available from manufacturers, they carried specific investigational limitations, since the FDA had not granted their approval. In 2001, FDA approval of a 635 nanometer (nm) laser to treat minor painful conditions renewed the United States' interest in low-energy lasers. This progress was followed in 2002 by FDA approval of a gallium-aluminum-arsenide (830 nm) low-energy laser to treat carpal tunnel syndrome.

Renewed interest in laser light therapy led to the clinical use of other forms of light therapy, including superluminous light emitting diode (SLED) devices. SLED devices do not require FDA approval, as they are considered superficial heating modalities, not laser devices. By contrast, low-energy laser devices are considered non-thermal or “cold laser.”

Today's devices use a laser diode to produce monochromatic, wavelength-specific light energy. The medium used determines the wavelength. For example, gallium, aluminum and arsenide in the diode configuration produce infrared light with a wavelength of 830 nm. Laser light travels as a highly columated and coherent beam of light energy with minimal divergence. In contrast, SLEDs produce light similar to a light bulb. Even if SLEDs produce light within a narrow range of wavelengths (color), this light doesn't have the properties of coherence and collimation.

Various studies demonstrate that laser light penetrates various cellular and sub-cellular layers to particular tissue depths, and other cellular and sub-cellular molecules absorb specific wavelengths of light to produce physiological changes. Since low-energy laser light is non-thermal, these physiological changes are the result of photobiostimulation, and not a heating effect.

In an article in the Journal of Biological Chemistry from 2004, researchers tested the efficacy of LEDs in therapeutic devices. Among the wavelengths tested there (670, 728, 770, nm and 830, and 880 nm), the most effective ones for the purpose of reversing the detrimental effect of tetrodotoxin were 670 nm and 830 nm. LEDs generate virtually no heat and thus pose no potential thermal injury during treatment. Additionally, LED emissions are well tolerated by biological tissues and have no known detrimental effect. As a therapeutic device, LED has achieved FDA non-significant risk status. Moreover, LED units are compact, portable and affordable.

Specific wavelength may be important for two reasons. First, wavelength is a principal factor that determines depth of tissue penetration. For example, most of the energy from infrared laser light (830 nm to 904 nm) is absorbed within 2 to 5 cm, which makes it effective for bones, joints and deep muscle problems. Helium-neon light energy (632.8 nm) is absorbed within 0.5 cm. Second, various cellular and molecular structures absorb and respond to specific wavelengths of light. For example, chromophores, photosensitive molecules within the membranes of cells and organelles, are sensitive to specific wavelengths. Visible red light at a wavelength of 660 nm penetrates tissue to a depth of about 0.8 to 1 cm, and is very beneficial in treating problems close to the skin, such as wounds, cuts, scars, and infection. Mitochondrial cytochromes absorb infrared light (700 nm to 1,200 nm), while myoglobin and hemoglobin absorb visible light, notably reds (600 nm to 700 nm).

The coherence of laser light allows concentrated bundles of light energy to reach target cells, even as its coherence breaks down as light penetrates the layers of cells and tissues. When cell components absorb light energy, chemical reactions within the cell may be facilitated, inhibited, or altered. The Arndt-Schultz law suggests that specific ranges of light energy dosages are required for effective biostimulation. Dosages that are too low or too high may have no effect or a negative effect.

Laser light dosage per stimulation point over the target structure is described as energy density, measured in joules/cm2. Energy density for a continuous laser light mode is determined by multiplying the mean power (watts) by the time (seconds) of application, divided by the beam's contact area (cm2) on the skin. Laser units vary in mean watts produced per diode. Therefore, clinicians may need to manually calculate the total dosage per stimulation point, unless the unit features preset dosage parameters for various conditions. Studies have revealed that an effective dosage range of 1.0 to 4.0 joules/cm2 can be effective for inflammatory conditions and pain syndromes.

As far as treating individuals, lasers hasten the inflammatory process through mitochondrial chromophore stimulation. This increases respiratory chain activity, which enhances ATP synthesis, cellular repair, reproduction, and formation of low amounts of reactive O₂ species (ROS), e.g. H₂O₂. This then leads to DNA and RNA synthesis; protein synthesis, mitosis and cell proliferation. For example, most cell stimulation increases histamine, which raises vasodilation. Serotonin and nitric oxide levels increase and lead to decreased tissue ischemia, increased perfusion and removal of cellular debris. Increased nitric oxide levels have also been reported to account for decreased bradykinin levels. Also, laser biostimulation raises beta-endorphin levels and suppresses depolarization of C-fiber pain afferents. The result is that the absorbed energy is used to repair the tissue, reduce pain, and restore normalcy to an otherwise impaired biological process.

The following benefits of laser light have been found: increased circulation through the formation of new capillaries to replace damaged ones; stimulated production of collagen, an essential protein in tissue repair and replacement; increased lymphatic system activity, which relieves swelling; stimulated release of adenosine triphosohate (ATP), allowing the cell to accept nutrients faster and get rid of waste products faster by increasing energy level in the cell; increased RNA and DNA synthesis which helps the body replace damages cells more quickly; reduced excitability of nervous tissue which relieves pain; stimulated fibroblastic activity, aiding in the repair of connective tissues; increased phagocytosis (scavenging and ingesting dead or degenerated cells), important in fighting infection before the healing process; induced thermal-like effect in the tissue by raising the temperature of the cells through a photochemical reaction; stimulated tissue granulation and connective tissue projections; stimulated acetylcholine release which causes cardiac inhibition, vasodilatation, gastrointestinal peristalsis, and other parasympathetic effects; and stimulated endorphins and enkephalins to facilitate long-term pain relief.

While the beneficial effects of laser light can translate to better clinical outcomes for a host of conditions, marketing any medical device with laser units requires FDA approval. Currently, the only condition approved for low-energy laser treatment by the FDA is carpal tunnel syndrome. Nevertheless, therapists routinely use lasers “off-label” to treat musculoskeletal conditions that are consistent with standards of practice. The rationale for treating carpal tunnel syndrome (alleviating pain, improving blood perfusion, and stimulating mitochondrial activity for tissue healing) is also consistent with the treatment of tarsal tunnel syndrome, lateral epicondylitis and trigger point pain syndromes.

However, it is important to note that laser biostimulation carries contraindications. While it enhances blood perfusion and increases metabolic activity, treatment over neoplastic tumors and active growth plates is contraindicated. Using irradiation over the thyroid gland or the uterus of a pregnant woman is also contraindicated. Therapists and patients should wear protective glasses to avoid eye exposure.

Using the code for infrared modality, where it may be accepted, requires that the modality provide superficial heat. However, low-energy laser is non-thermal in the nature of its energy production and intended purpose. Some clinicians have reported success using the unlisted modality or unlisted procedure codes, following prior negotiation with a patient. Many patients pay out-of-pocket for the modality. Additional research into clinical effectiveness is necessary before an assigned code for low-energy laser can be granted.

The bulk of the research into laser's therapeutic effects has been conducted in Canada, Europe, Asia, Australia and New Zealand, which have longer histories of laser use than the United States. Most clinical research has examined low-energy lasers and not other forms of light therapy such as SLEDs. Clinicians should be cautious about extrapolating findings from laser research to these related forms of non-laser light therapy.

A review of clinical trials suggests that the evidence of effectiveness to treat musculoskeletal conditions is often equivocal. The strongest evidence supports the use of low-energy lasers to treat myofascial trigger point pain syndromes and similar conditions that result in pain and inflammation. Patients with carpal tunnel syndrome tend to improve most dramatically when their symptoms are mild to moderate.

As with any emerging treatment, there's an essential need to collect clinical effectiveness data that supports laser therapy. Also, it is crucial to accurately document treatment parameters for the clinic chart and for systematic data collection. This data should include information on precise, marked treatment locations on the patient, laser type and wavelength, continuous or pulsed delivery, and dosage delivered per site. Currently, there's no treatment code assigned for laser therapy. As such, clinicians often use laser treatment as an adjunct modality to support other procedures, such as manual therapy or therapeutic exercise.

It would be advantageous to have an apparatus capable of delivering EMR therapy during use of a mouse or other computer peripheral. Accordingly, there is a need to provide computer peripherals such as a mouse, joystick, foot stick etc. for users who have and/or want to prevent body (e.g. hand, feet, etc.) injuries or maladies.

### DEFINITION OF CLAIM TERMS

The following terms are used in the claims of the patent as filed and are intended to have their broadest meaning consistent with the requirements of law. Where alternative meanings are possible, the broadest meaning is intended. All words used in the claims are intended to be used in the normal, customary usage of grammar and the English language.

“Computer pointing device” means any input device that is used in conjunction with a computer to translate motion of the device (e.g., 2-D or 3-D motion) into motion (e.g., cursor motion or field-of-view motion) on a viewing (e.g., computer, LED, plasma, projection) screen. For example, computer pointing devices may include a computer mouse, a game controller, a joystick, etc.

“Hand” means any part of a user's lower arm portion that may come in contact with the device during use.

“Keyboard” means any device, whether electronically controlled or not, with manually manipulable keys, such as a computer keyboard (whether desktop, laptop, PDA, etc.), electronic cash register, etc.

“Light-emitting diode (LED)” means a semiconductor diode that emits light when an electric current is applied to it.

“Repetitive Stress Injury (RSI)” means any injury, ailment, and/or malady caused in whole or in part by or exacerbated by a computer pointing device, including but not limited to a computer mouse.

“Wrist” means the area of a user's hand that includes, but is not limited to the transverse carpal ligament and median nerve.

### SUMMARY OF THE INVENTION

The objects mentioned above, as well as other objects, are solved by the present invention, which overcomes disadvantages of commonly used computer pointing devices, while providing new advantages not previously obtainable with such computer pointing devices.

The present invention provides a keyboard for promoting healing from repetitive stress injuries that overcomes the aforementioned drawbacks. In a preferred embodiment, the keyboard includes a body housing manually manipulable keys, and one or more devices (e.g., LEDs, SLEDs, cold lasers, etc.) located either on the keyboard or on an attachment thereto, and configured to emit electromagnetic radiation (EMR) toward a user's wrist and hand at wavelengths of between about 600 Nanometers and 1000 Nanometers when the user's hand is positioned to allow the fingers to depress the keys, in order to facilitate the treatment of repetitive stress injury.

The EMR-emitting device(s) may include a plurality of LEDs designed to emit EMR at certain preferred wavelengths, including but not limited to: between about 632 Nanometers and 635 Nanometers; 670 Nanometers; 780 Nanometers; between 870 Nanometers and 890 Nanometers; and 904 Nanometers.

According to another aspect of the invention, the plurality of LEDs may constitute low-energy lasers to treat myofascial trigger point pain syndromes and similar conditions that result in pain and inflammation. In yet another aspect of the invention the plurality of LEDs may constitute lasers which hasten the inflammatory process through mitochondrial chromophore stimulation. The lasers, through biostimulation, may raise beta-endorphin levels and suppress depolarization of C-fiber pain afferents.

In accordance with another aspect of the invention, the EMR-emitting device(s) may constitute a plurality of LEDs emitting EMR at a predetermined energy density. In one preferred range, the energy density constitutes a dosage range of about 1.0 to 4.0 joules/cm².

According to another aspect of the invention, the EMR-emitting device(s) may be located on the keyboard or an attachment thereto, and configured to emit EMR for a predetermined duration. These emissions may constitute periodic “pulses” in which EMR is emitted for a period of time (e.g., 10 minutes), then stopped for a period of time (e.g., 10 minutes), then pulsed again for a period of time, etc. Light, audio or other indications may be given to the user to indicate that EMR emissions are occurring or are not occurring. The EMR-emitting device(s) may also periodically pulse at a much faster rate, measured in seconds.

Appropriate software in electrical communication with a graphical user interface can enable user control over the intensity and/or duration of the EMR emissions, such as but not limited to providing functionality similar to a volume control on a computer monitor. Mechanical actuators such as wheels, buttons or levers may be located on the keyboard or elsewhere for this purpose, as well.

In accordance with another aspect of the invention, a method of using the above-described keyboard is also disclosed. Such a keyboard or its attachment may include a bank of EMR-emitting devices, such as a bank or array of LEDs, positioned to emit EMR toward a user's wrist and hand when the user's hand is positioned to allow the fingers to depress the keys of the keyboard. In accordance with another aspect of the invention, the method may also include attaching a transparent or translucent window to a portion of the keyboard body and over the bank or array of LEDs, to enable the LEDs to emit EMR which will penetrate to the user's wrist and/or hand.

Various other features and advantages of the present invention will be apparent from the following detailed description and the drawings.

### BRIEF DESCRIPTION OF THE DRAWINGS

The novel features which are characteristic of the invention are set forth in the appended claims. The invention itself, however, together with further objects and attendant advantages thereof, will be best understood by reference to the following description taken in connection with the accompanying drawings, in which:

FIG. 1 is a perspective view of on embodiment of a computer pointing device;

FIG. 2 is a side view of the computer pointing device of FIG. 1

FIG. 3a is a cross-sectional view along line 3-3 of FIG. 1 according to one embodiment of the present invention;

FIG. 3b is a cross-sectional view along line 3-3 of FIG. 1 according to another embodiment of the present invention;

FIG. 4 is a cutaway view of the computer pointing device of FIG. 1;

FIG. 5 is a schematic block diagram of a computer system incorporating the computer pointing device of FIG. 1;

FIG. 6 is a left-side and top perspective view of a preferred embodiment of the pointing device;

FIG. 7 is a top, perspective view of the pointing device shown in FIG. 6;

FIG. 8 is a left-side, perspective view of the pointing device shown in FIG. 6;

FIG. 9 is a perspective view of the hand of a computer pointing device use;

FIG. 10 is a left-side, perspective view of the pointing device shown in FIG. 6 during use;

FIG. 11 is a perspective view of one preferred embodiment of a keyboard with LEDs emitting EMR according to the present invention;

FIG. 12 is a partial, enlarged perspective view of an alternative embodiment of the keyboard invention, and includes a lower portion of a keyboard attachment partially exposed; and

FIG. 13 is a side, partially sectioned, view of the keyboard attachment shown in FIG. 12.

The components in the drawings are not necessarily to scale, emphasis instead being placed upon clearly illustrating the principles of the present invention.

### DETAILED DESCRIPTION OF THE PREFERRED EMBODIMENT

Set forth below is a description of what are believed to be the preferred embodiments and/or best examples of the invention claimed. Future and present alternatives and modifications to this preferred embodiment are contemplated. Any alternatives or modifications which make insubstantial changes in function, in purpose, in structure, or in result are intended to be covered by the claims of this patent.

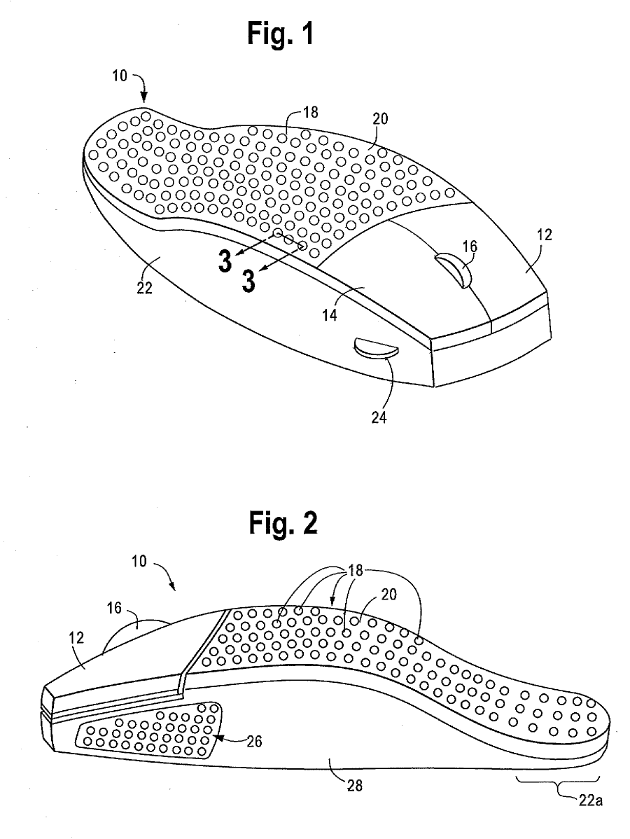

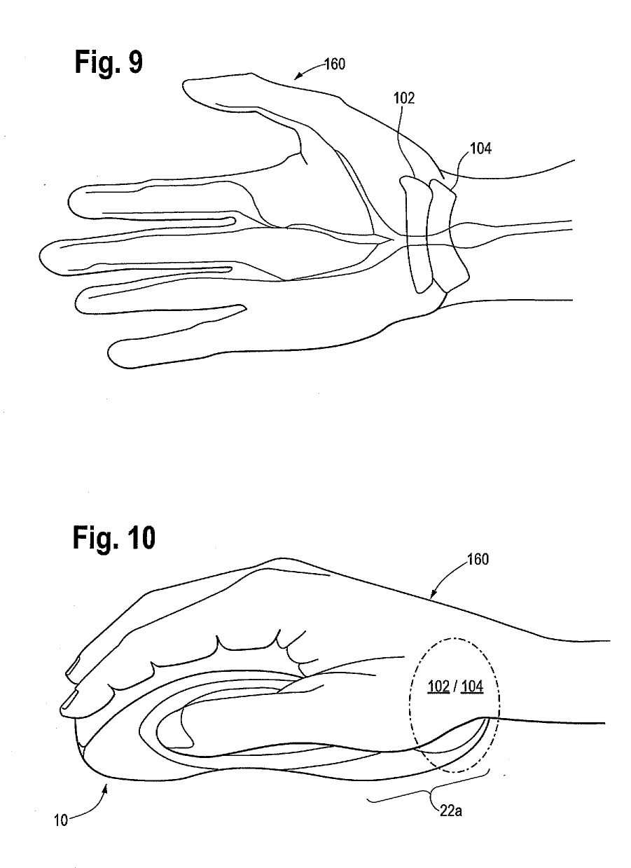

Referring to first to FIGS. 1 and 2, a preferred embodiment of a computer pointing device, generally designated by reference numeral 10, such as a computer mouse, is shown, for controlling a computer cursor (not shown). Pointing device 10 may include a body 22, a left button 12, right button 14, and scroll wheel 16 for performing various functions that input information to the computer. In this preferred embodiment, the rear portion of body may include an extended tail portion 22a, which may extends so as to support the user's hand at his or her wrist area, such that the extended tail 22a may hit the transverse carpal ligament 102 and/or median nerve 104 of the user's hand 160 as shown in FIGS. 9-10. An EMR-emitting device, such as a bank or plurality of EMR-emitting LEDs 18, 26, may be attached to handheld computer pointing device 10. LEDs 18 may be positioned adjacent a top portion 20 of body 22 of pointing device 10 so as to direct EMR, such as visible and/or infrared light, toward a user's hand when using pointing device 10. LEDs 18 may be configured to emit a variety of light wavelengths, such as between about 600-1000 Nanometers, or various preferred wavelengths, such as: 632 Nanometers; 633 Nanometers; 635 Nanometers; 670 Nanometers; 780 Nanometers; 830 Nanometers; 880 Nanometers; 890 Nanometers; and 904 Nanometers. LED bank 18 may also include different arrays of LEDS; for example, a first array or group of LEDs emitting light at a first wavelength may be employed, and a second array or group of LEDs emitting light at a second wavelength may also be employed. Additional arrays or groups emitting additional wavelengths may also be used.

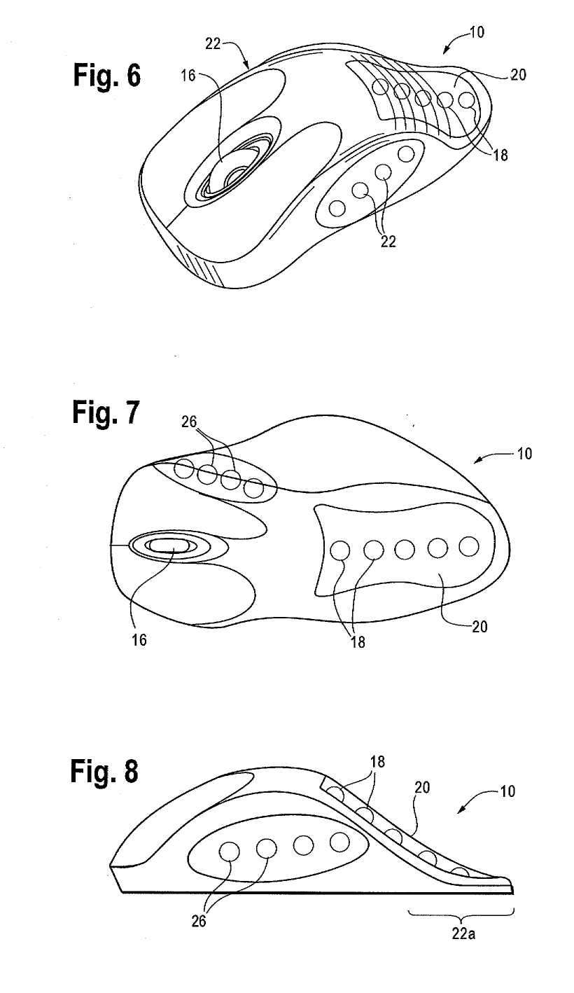

Referring to FIGS. 6-8, an alternative embodiment of a computer mouse 10 is shown, including a body 22, a wheel 16, a top LED array 18, side LED arrays 16, and an extended tail portion 22 (FIG. 8).

Pointing device 10 preferably includes a switch 24 (FIG. 1) for manually setting a powered state of the EMR-emitting device such as the plurality of LEDs 18. Switch 24 may be an analog or a digital switch. Alternatively or in addition to switch 24, as will be described below, a computer program may be provided to predetermine and automatically control the powered state of the EMR-emitting device, such as the plurality of LEDs 18, 26.

One skilled in the art will recognize that, given functionality needs, different computer pointing device technology may be employed from that shown in FIG. 1. However, it is contemplated that a computer pointing device having different functionality from that described herein is possible and within the scope of the appended claims. In addition to different computer mice with different functionalities, other pointing devices such as joysticks, game controllers, keyboards (as further discussed below), etc., may be outfitted with EMR-emitting devices as disclosed here to provide therapeutic affects as described here.

Referring now to FIG. 2, pointing device 10 may include a plurality of LEDs 26 positioned adjacent to a side portion 28 of body 22. The plurality of LEDs 26 may be positioned so as to direct EMR toward a thumb of the user's hand when using pointing device 10.

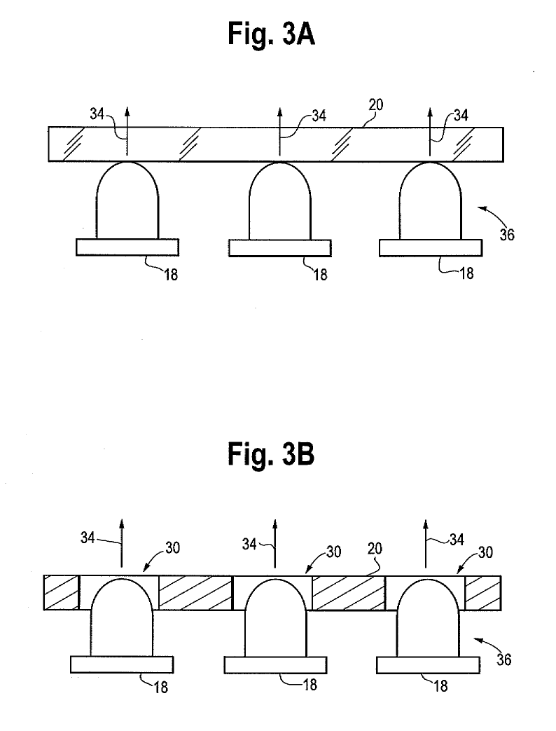

Referring to FIGS. 3a and 3b, two embodiments of a cross-sectional view along line 3-3 of FIG. 1 are shown. In one embodiment, as shown in FIG. 3a, at least a portion of top portion 20 is constructed of a material translucent to light 34. The plurality of LEDs 18 may be positioned within an interior volume 36 of pointing device 10 and positioned to emit light 34 exterior to pointing device 10 toward a palm of a user's hand.

In another embodiment, shown in FIG. 3b, a plurality of apertures 30 may be formed in top portion 20 of pointing device 10. The plurality of LEDs 18, 26 may be aligned with the plurality of apertures 30 and may extend into or through the plurality of apertures 30, emitting infrared light directly from the pointing device to the hand of a user.

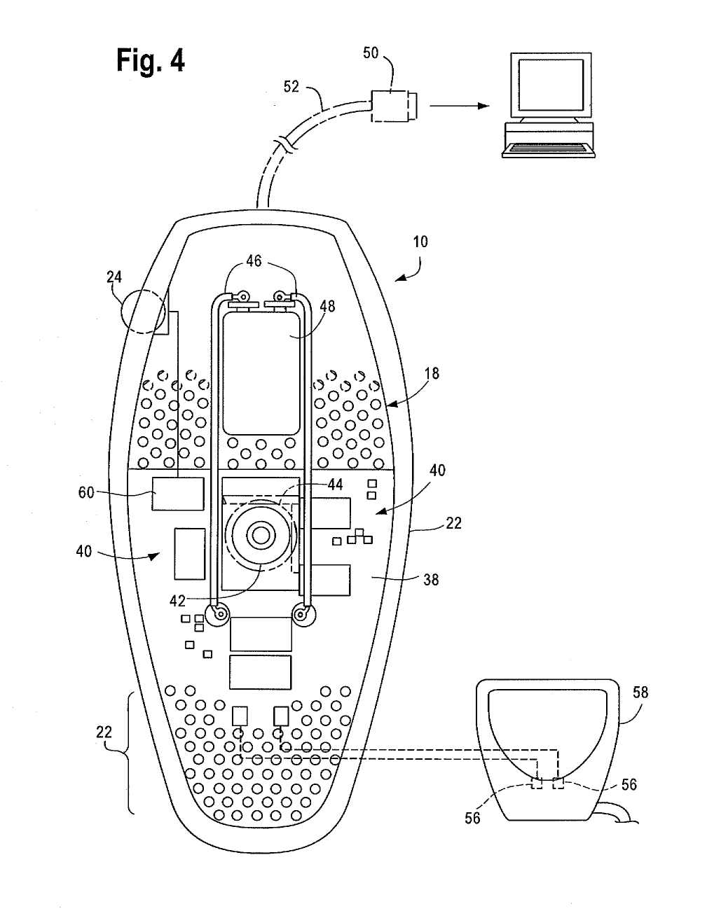

Referring to FIG. 4, a cutaway view of the bottom of the pointing device 10 is shown. The pointing device 10 may include an electronics board 38 having a plurality of electronic components 40 connected thereto to provide the electrical functions (e.g., LED/rollberball/button electronic communication) of pointing device 10, as is well known in the art. A movement sensing device 42 (e.g., trackball, optical sensing device, etc.) may be employed to determine the two-dimensional movement of pointing device 10 relative to a mouse support surface such as a desk (not shown). In a preferred embodiment, movement sensing device 42 may constitute an optical transceiver comprising an LED emitter and a receiver to sense reflected light from the LED emitter. As one specific example, movement sensing device 42 may be a ball 44 configured to optically or mechanically sense a movement direction of pointing device 10. Electrical functions of the left button 12, right button 14, and scroll wheel 16 may also be provided via the plurality of electronic components 40.

Still referring to FIG. 4, a power bus 46 may be employed to provide electrical power to the plurality of electronics 40 and to the plurality of LEDs 18. Voltage on the power bus 46 may be supplied by a battery 48. Alternatively, voltage on the power bus 46 may be supplied via a USB connector 50 and cable 52 connected to a computer power supply (not shown). In a preferred embodiment, battery 48 may be a rechargeable battery. A pair of contacts 54 may be connected to the power bus 46, and coupled with a pair of contacts 56 to a docking station 58. The docking station 58 may be used to provide recharging current to the rechargeable battery 48 to recharge the rechargeable battery 48 when the peripheral 10 is not in use.

Still referring to FIG. 4, peripheral 10 may include a controller 60 configured to control a powered state of the plurality of LEDs 18, 26. In a preferred embodiment, controller 60 may be configured to control the on-and-off powered state of the plurality of LEDs 18, 26. Switch 24 may be connected to controller 60, allowing a user to input a desired powered state of the plurality of LEDs 18, 26. Controller 60 may be configured to control an on-and-off powered state of each LED 18 individually. In this manner, the desired powered status may indicate that the controller 60 turn on all LEDs 18, 26, turn off all LEDs 18, 26, or control the LEDs 18, 26 according to a predefined pattern. It is contemplated however, that controller 60 may be configured to control the powered state of a group or groups of LEDs 18 as a whole.

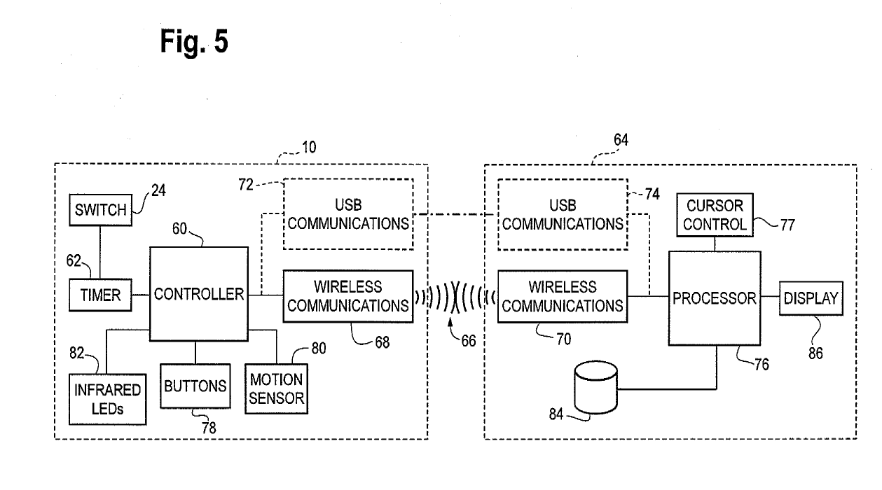

FIG. 5 shows a schematic block diagram of a computer 64 preferably connected to handheld computer pointing device 10 via a wireless communications link 66 between a wireless communication circuit 68 of pointing device 10 and a wireless communication circuit 70 of computer 64. Alternatively, pointing device 10 and computer 64 may be interconnected and communicate via USB communication circuits 72, 74. Controller 60 may be configured to send cursor commands via wireless communications link 66 to processor 76 of computer 64 for cursor commands such as button clicks and cursor coordinate modification. Cursor commands sent to a cursor control 77 may be determined from buttons 78 and motion sensor 80.

In a preferred embodiment, switch 24 may include a number of intermediate positions that may be used to set a timer 62. Preferably, timer 62 controls a time in which an individual or a group of LEDs 82 remain in a particular powered state. For example, timer 62 may cause controller 60 to toggle the powered state of a group of LEDs 82 to an off state upon the expiration of a time threshold. Alternatively, timer 62 may set a sequence delay time between the off state of one LED and the on state of another infrared LED.

Controller 60 may set timer 62 according to a particular predefined pattern. For example, the predefined pattern may include turning on a group of LEDs 82 and, after timer 62 reaches a threshold, turning off the group of LEDs 82. The predefined pattern may also indicate a delay time between the turning off of a group of one or more LEDs 82 and the turning on of another group.

Referring still to FIG. 5, alternatively or in addition to controller 60 controlling the powered states of LEDs 82, computer 64 may programmed to control the powered states of LEDs 82 via wireless communications link 66. A computer program stored in memory 84 may cause the computer 64 to transmit instructions to handheld computer pointing device 10 causing the powered states of the LEDs 82 to toggle between on and off states. Display 86 may include a graphical user interface (not shown) allowing the user to set, via keyboard or cursor input, the on state, off state, and predefined pattern of LEDs 82.

Referring to FIGS. 9 and 10, the user's hand 160 is shown. The user's transverse carpal ligament 102 and median nerve 104 can be seen. When mouse 10 of the present invention is used, the user's hand 160 is positioned generally as shown in FIG. 10, such that the extended tail 22 of mouse 10 is positioned adjacent ligament 102 and nerve 104, so that EMR emissions can be directed to facilitate the treatment of repetitive stress injury.

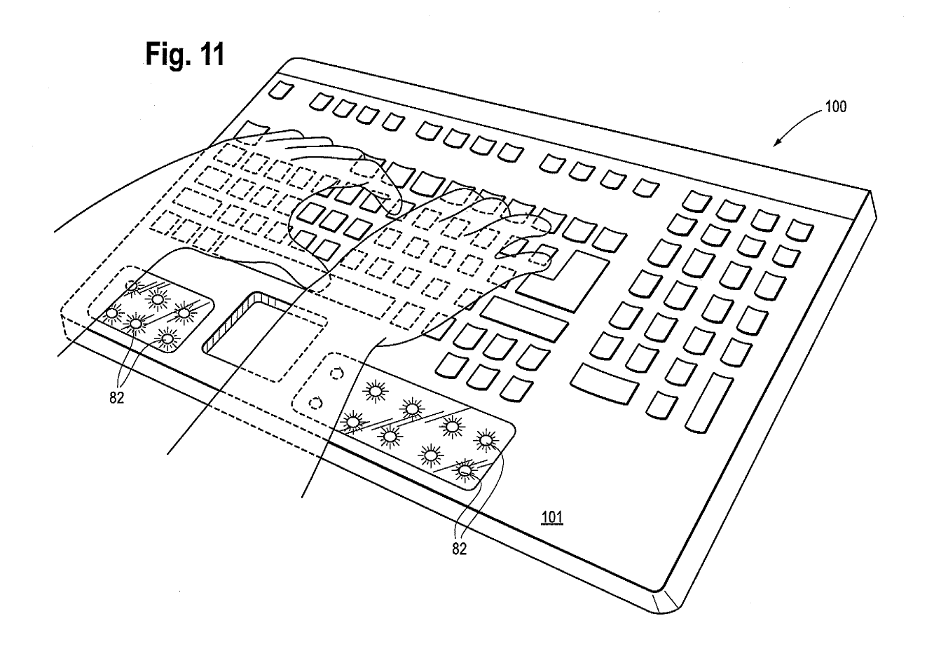

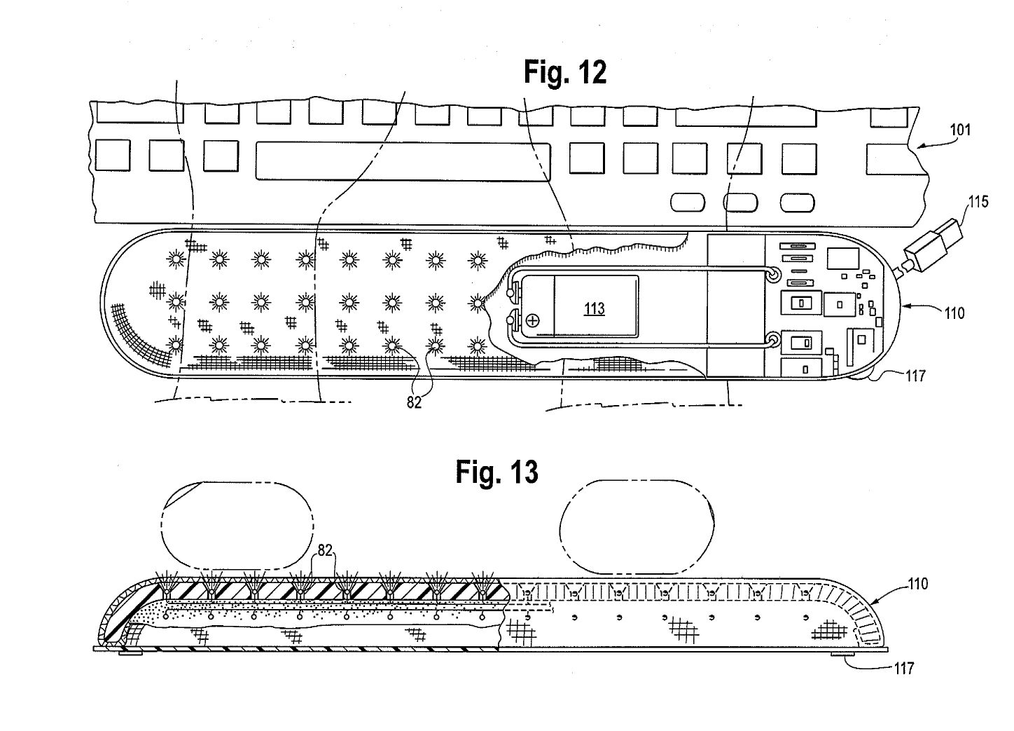

Referring now to FIG. 11, a keyboard 100, which may be used with a computer, processor or other device (e.g., electronic cash register), may be manufactured with LEDs 82 emitting EMR in the intensity ranges (e.g., wavelength of 600-1000 nanometers), fluence, and the constant and/or periodic durations described above. LEDs 82 may be built into the bottom of the keyboard, which may have a clear portion on the top surface to allow the EMRs to penetrate the wrist/upper hand area during keyboard use, such as the carpal tunnel area of the wrist/hand. The intensity and/or duration of the EMR may also be controlled as described above, through keyboard, mouse and cursor control and/or through use of a graphical user interface on a screen/monitor. The EMR may be emitted from an elongated portion of a specially manufactured computer or laptop keyboard 101, as shown in FIG. 11; alternatively, the EMR may be emitted from LEDs 82 on a keyboard/wristboard attachment 110 to a conventional computer or laptop keyboard, as shown in FIG. 12; attachment 110 may be designed to be snap-fit or otherwise connected and/or retrofitted to an existing, conventional keyboard 101. Attachment 110 may include battery 113, USB connector 115, and an electric current adjustment wheel 117.

Wheel 117 may be used to adjust the intensity of the LED EMRs, for example. Another wheel or other control may be used to control the duration of the EMRs, as well. A graphical user interface, in communication with suitable software, could be used to facilitate such control, such as by providing visual controls for intensity and/or duration similar to a volume control on a computer monitor.

The keyboard areas and/or wrist pad may house a bank of photobiomodulation lights which may run the length of the desktop or laptop keyboard and/or wrist pad areas.

The ER emissions are preferably non-thermal in nature. Inventor testing has not indicated that ER emissions delivering heat have a beneficial impact on joint stress/injury. For this reason it is not believed that there is any need to use heat of any substantial amount. This will avoid any danger of thermal burns; further, heat has a deleterious impact on electrical systems.

The invention, as described above, may be used to stimulate the blood flow in a user's hand to aid in reducing inflammation and pain. The user will thus be able to re-energize the body/hand at the cellular level, promoting a dispersion of pain and preserving the prophylactic effects of, and the dramatic increase in, blood flow in the region stimulated by the EMR emissions. By combining the functions of a computer pointing device and/or keyboard with that of EMR therapy, computer users may reduce pain, promote healing, and provide a curative methodology to their body/hand without the need to stop working, gaming, etc. for a session of therapy.

Those of ordinary skill in the art will appreciate that, while the use of LEDs has been described as a cost-effective and safe EMR-emitting device, other such devices may be used and may be found effective, including lasers, SLEDs, etc.

The above description is not intended to limit the meaning of the words used in the following claims that define the invention. For example, while preferred embodiments have been described above, persons of ordinary skill in the art will understand that a variety of other designs still falling within the scope of the following claims may be envisioned and used. It is contemplated that future modifications in structure, function or result will exist that are not substantial changes and that all such insubstantial changes in what is claimed are intended to be covered by the claims.

I claim:

1. A keyboard, comprising:
a body including manually manipulable keys;
one or more devices incorporated in or attached to the body for emitting electromagnetic radiation (EMR) toward a user's wrist or hand at wavelengths of between about 600 Nanometers and 1000 Nanometers, to facilitate the treatment of repetitive stress injury when the user's hand is positioned to move the body relative to the reference point.

2. The keyboard of claim 1, wherein the one or more EMR-emitting devices comprise a plurality of LEDs.

3. The keyboard of claim 1, wherein the one or more EMR-emitting devices emits wavelengths in at least one of the following ranges: 632-635 nm; 670 nm; 780 nm; 870-890 nm; and 904 nm.

4. The keyboard of claim 1, wherein the one or more EMR-emitting devices comprise one or more low-energy lasers to treat myofascial trigger point pain syndromes and similar conditions that result in pain and inflammation.

5. The keyboard of claim 4, wherein the one or more lasers hasten the inflammatory process through mitochondrial chromophore stimulation.

6. The keyboard of claim 4, wherein the one or more lasers through biostimulation raise beta-endorphin levels and suppress depolarization of C-fiber pain afferents.

7. The keyboard of claim 1, wherein the one or more EMR-emitting devices are configured to emit EMR at a predetermined energy density.

8. The keyboard of claim 8, wherein the predetermined energy density is in a dosage range of about 1.0 to 4.0 joules/cm².

9. The keyboard of claim 1, wherein the one or more EMR-emitting devices are configured to emit EMR for a predetermined duration.

10. The keyboard of claim 9, wherein the one or more EMR-emitting devices are configured to provide EMR emissions for a first predetermined duration, to stop EMR emissions for a second predetermined duration, and then to provide EMR emissions for a third predetermined duration which may be the same as or different than the first predetermined duration.

11. The keyboard of claim 2, wherein the plurality of LEDs comprises at least two arrays of LEDs emitting different ranges of wavelengths.

12. The keyboard of claim 2, wherein the plurality of LEDs periodically pulse with EMR emissions.

13. The keyboard of claim 1, wherein a computer program is in electrical communication with the one or more EMR-emitting devices to facilitate control of the intensity or duration of emissions from the devices.

14. The keyboard of claim 13, wherein the computer program is in electrical communication with a graphical user interface for facilitating control over the intensity or duration of emissions from the EMR-emitting device.

15. The keyboard of claim 1, wherein the keyboard includes a cover for the one or more EMR-emitting devices that allows the EMR to pass through the cover and to the user's wrist and hand.

16. The keyboard of claim 15, wherein the cover is transparent or translucent.

17. The keyboard of claim 1, further comprising a mechanical actuator located on the keyboard for adjusting the intensity or duration of the EMR.

18. The keyboard of claim 17, wherein the mechanical actuator comprises a wheel.

19. A method of using a keyboard for emitting EMR to a user's wrist and hand, comprising the steps of
providing a keyboard with a body housing keys and one or more EMR-emitting devices positioned so that when the user utilizes the keyboard, the user's wrist and hand are located adjacent the one or more EMR-emitting devices; and
causing the EMR-emitting device to activate when the keyboard is used, thereby exposing the user's wrist and hand to EMR in order to prevent, or facilitate the treatment of, repetitive stress injury.

20. The method of claim 15, wherein the one or more EMR-emitting devices comprise a plurality of LEDs.
